(19) United States
(12) Patent Application Publication
(10) Pub. No.: US 2026/0219257 A1
(43) Pub. Date: Jul. 30, 2026

(54) METHOD AND APPARATUS FOR BREATH SCREENING FOR TUBERCULOSIS

(71) Applicant: ANIKA STERILIS PRIVATE LIMITED, Gurugram (IN)

(72) Inventor: Sabatino NACSON, Thornhill, Ontario (CA)

(21) Appl. No.: 19/132,321

(22) PCT Filed: Nov. 22, 2023
(86) PCT No.: PCT/IB2023/061765

§ 371 Date: May 22, 2025

Related U.S. Application Data
(60) Provisional application No. 63/427,387, filed on Nov. 22, 2022.

(51) Int. Cl. G01N 33/497; A61B 5/08; A61B 5/097; G01N 30/72

(52) U.S. Cl. CPC G01N 33/4975; A61B 5/082; A61B 5/097; G01N 30/7206

(57) ABSTRACT

The present disclosure relates to a method of detecting tuberculosis infection, the method comprising collecting a breath sample from a subject, and detecting in the breath sample at least one tuberculosis-infection-indicative volatile organic compounds selected from a group consisting of C₅H₁₀O aldehyde, C₇H₁₄O aliphatic ketone, C₉H₁₈O aliphatic ketone, C₈H₁₆O aldehyde, C₈H₁₈O alkyl alcohol, C₉H₁₂ alkyl aromatic, C₁₂H₂₆O alkyl alcohol, C₉H₁₈O aliphatic aldehyde, C₁₀H₁₄ alkyl aromatic, C₇H₇NO₂ carboxylate alkyl pyridine, C₈H₁₆ alkyl cyclohexane, and any combination thereof. The present disclosure also relates to an apparatus for detecting tuberculosis infection in a subject, the apparatus comprising a breath sample receiver and a detector associated with the breath sample receiver for detecting at least one tuberculosis-infection-indicative volatile organic compound in a breath sample.

### TECHNICAL FIELD

The present disclosure relates to a method and apparatus for breath screening for tuberculosis.

### BACKGROUND

In recent years, tuberculosis (TB) infections have been increasing worldwide, and have started to reappear in developed countries from which the infectious disease had largely disappeared.

The presence of some lung-related diseases, including TB, can be determined using biomarker(s) in a subject's breath. Some of these biomarkers are known in the art and have been documented in scientific journal publications including Phillips et al “Point-of-care breath test for biomarkers of active pulmonary tuberculosis” Tuberculosis 92 (2012) 314-320, Phillips et al “Volatile biomarkers of pulmonary tuberculosis in the breath” Tuberculosis (2007) 87, 44-52, Phillips et al “Breath biomarkers of active pulmonary tuberculosis”, Tuberculosis 90 (2010), 145-151, Kolk et al “Breath-based biomarkers for tuberculosis”, Proc. Of SPIE Vol. 8371, 2012, 83710A-1-9, Bobak et al “Breath can discriminate tuberculosis from other lower respiratory illness in children”, Nature scientific reports 2021, 11:2704, Pal et al “Breathe Analysis in tuberculosis disease recognition in new millennium”, RRJMB Volume 2, issue 3, July-September 2013, Zacharia et al “GCMS based detection of lipid biomarkers of Mycobacterium tuberculosis in the serum specimen”, J. of Respiratory Res. 2016 Jun. 2 (2): 47-55, Kolk et al “Breath analysis as a potential diagnostic tool for tuberculosis”, Int. J. Tuberc Lung Dis 16 (6): 777-782, 2012, Belizario et al “Breath biopsy and discovery of exclusive volatile organic compounds for diagnosis of infectious diseases”, Frontiers in Cellular and Infection Microbiology, January 2021, Volume 10, article 564194, and Nizio et al “In vitro volatile organic compounds profiling using GCxGC-TOFMS to differentiate bacteria associated with lung infections: a proof-of-concept study”, J. Breath Res. 10 (2016), 026008.

For subjects suffering from pulmonary tuberculosis, the volatile organic compounds (VOCs) in their breath samples may be altered by the presence of Mycobacteria and oxidative stress. Such alteration may result in a distinctive VOC profile. It has been reported that several species of Mycobacteria produce VOC metabolites that act as chemical “fingerprints”. For example, patients with TB often suffer from increased oxidative stress, as reported in their associated serum markers including lipid peroxidation products, conjugated dienes, malondialdehyde, and allantoin, which are generally increased in patients with active TB. Oxidative stress also liberates distinctive VOCs into the breath samples of subjects, including aldehydes, alkyl alcohols, aromatic hydrocarbons, and others.

U.S. Pat. Nos. 9,170,232 and 9,541,525 to Nacson et al describe an ion-mobility spectrometer with front fast GC separation of the sample analytes. U.S. Pat. No. 9,329,156 to Nacson describes a filter used in the collection of sample breath and enrichment of the sample. U.S. Pat. No. 5,395,589 to Nacson discloses an apparatus for preconcentrating trace amounts of organic vapors in a sample of air for subsequent detection. Where permissible according to local patent laws, these patents are incorporated herein by reference.

Early detection of lung-related diseases, including TB, may result in lives saved and an overall decrease in healthcare expenditures.

### SUMMARY

The present disclosure relates to a method and apparatus for breath screening for tuberculosis. It is an object of the disclosed method and apparatus to provide a means for straightforward, rapid and reasonably accurate testing for tuberculosis and related infectious diseases.

According to an aspect of the present disclosure, there is an apparatus for detecting tuberculosis infection in a subject, the apparatus comprising a breath sample receiver and a detector associated with the breath sample receiver for detecting at least one tuberculosis-infection-indicative volatile organic compound in a breath sample, the at least one tuberculosis-infection-indicative volatile organic compound selected from a group consisting of C₅H₁₀O aldehyde, C₇H₁₄O aliphatic ketone, C₉H₁₈O aliphatic ketone, C₈H₁₆O aldehyde, C₈H₁₈O alkyl alcohol, C₉H₁₂ alkyl aromatic, C₁₂H₂₆O alkyl alcohol, C₉H₁₈O aliphatic aldehyde, C₁₀H₁₄ alkyl aromatic, C₇H₇NO₂ carboxylate alkyl pyridine, C₈H₁₆ alkyl cyclohexane, and any combination thereof.

The breath sample receiver may be capable of receiving a nanocarbon treated sample card containing the breath sample. The nanocarbon treated sample card may further comprise a filter for separating the at least one tuberculosis-infection-indicative volatile organic compound in the breath sample from other volatile organic compounds that are not of interest in a determination of tuberculosis infection. The filter may be a charcoal filter.

The apparatus may further comprise a gas chromatographic unit. The detector may be an ion mobility spectrometer. The apparatus may further comprise a retention-time separation-analysis program for analyzing the at least one tuberculosis-infection-indicative volatile organic compound in the breath sample. The apparatus may further comprise a derivative-based-retention-time separation program for analyzing the at least one tuberculosis-infection-indicative volatile organic compound in the breath sample. The apparatus may further comprise a desorber unit.

According to another aspect of the present disclosure, there is a method of detecting tuberculosis infection in a subject, the method comprising collecting a breath sample from the subject, and detecting in the breath sample at least one tuberculosis-infection-indicative volatile organic compounds selected from a group consisting of C₅H₁₀O aldehyde, C₇H₁₄O aliphatic ketone, C₉H₁₈O aliphatic ketone, C₈H₁₆O aldehyde, C₈H₁₈O alkyl alcohol, C₉H₁₂ alkyl aromatic, C₁₂H₂₆O alkyl alcohol, C₉H₁₈O aliphatic aldehyde, C₁₀H₁₄ alkyl aromatic, C₇H₇NO₂ carboxylate alkyl pyridine, C₈H₁₆ alkyl cyclohexane, and any combination thereof.

The at least one tuberculosis-infection-indicative volatile organic compounds of the breath sample may be collected on a nanocarbon treated sample card. The nanocarbon treated sample card may comprise a filter for separating the at least one tuberculosis-infection-indicative volatile organic compound in the breath sample from other volatile organic compounds that are not of interest in a determination of tuberculosis infection.

The method may further comprise inserting the nanocarbon treated sample card into the breath sample receiver of an apparatus. The method may further comprise separating the at least one tuberculosis-infection-indicative volatile organic compounds by gas chromatography.

The method may further comprise detecting the at least one TB-infection-indicative volatile organic compounds by an ion mobility spectrometer. The method may further comprise analyzing the at least one TB-infection-indicative volatile organic compounds detected by the ion mobility spectrometer by using one of a retention-time separation-analysis program and a derivative-based-retention-time separation program.

This summary does not necessarily describe the entire scope of all aspects of the disclosure. Other aspects, features and advantages will be apparent to persons of ordinary skill in the art upon review of the following description of specific embodiments.

### DETAILED DESCRIPTION

Directional terms such as “top”, “bottom”, “upwards”, “downwards”, “vertically”, and “laterally” are used in the following description for the purpose of providing relative reference only, and are not intended to suggest any limitations on how any article is to be positioned during use, or to be mounted in an assembly or relative to an environment. Any element expressed in the singular form also encompasses its plural form. Any element expressed in the plural form also encompasses its singular form. The use of the word “a” or “an” when used herein in conjunction with the term “comprising” may mean “one”, but it is also consistent with the meaning of “one or more”, “at least one”, and “one or more than one”.

As used herein, the terms “comprising”, “having”, “including”, and “containing”, and grammatical variations thereof, are inclusive or open-ended and do not exclude additional, un-recited elements and/or method steps. The term “consisting essentially of” when used herein in connection with a composition, use or method, denotes that additional elements, method steps or both additional elements and method steps may be present, but that these additions do not materially affect the manner in which the recited composition, method or use functions. The term “consisting of” when used herein in connection with a composition, use or method, excludes the presence of additional elements and/or method steps.

The present disclosure relates to a method and apparatus for breath screening for tuberculosis. According to an embodiment of the apparatus, there is a breathalyzer comprising a gas chromatography (GC) unit and a ion mobility spectrometer (IMS). The IMS is coupled to the GC, and can be any IMS that is known in the art. A non-limiting example of an IMS in the art is described in U.S. Pat. No. 9,170,232. Also as contemplated in this embodiment, the breathalyzer comprises a front internal separator unit for use in thermal vaporization of a breath sample.

As contemplated in this embodiment, the breath sample is collected on a nanocarbon treated sample card. The nanocarbon treated sample card is inserted into a heated desorber module of the breathalyzer for vaporizing the collected sample into VOCs. These VOCs are separated by gas chromatography after which these VOCs are directed into the ionization source of the IMS for characterizing and detecting the VOC profile of these VOCs evolving from the breath sample collected on the nanocarbon treated sample card.

The VOC profile characterized and detected from the vaporized collected sample is compared against a library of profiles that have been uploaded onto the breathalyzer. As contemplated in this embodiment, the key VOCs for detecting tuberculosis infection are provided in Table 1 below:

TABLE 1

| Channel Designation | TYPE |
|---|---|
| TB1P, TB2P | C₅H₁₀O aldehyde |
| TB3P, TB3N | C₇H₁₄O aliphatic ketone |
| TB4P, TB4N | C₉H₁₈O aliphatic ketone |
| TB5P, TB5N | C₈H₁₆O aldehyde |
| TB6P, TB7P | C₈H₁₈O Alkyl alcohol |
| TB8P | C₉H₁₂ Alkyl aromatic |
| TB9P, TB9N | C₁₂H₂₆O Alkyl alcohol |
| TB10P, TB10N | C₉H₁₈O Aliphatic aldehyde |
| TB11P, TB12P | C₁₀H₁₄ Alkyl aromatic |
| TB13P, TB14P, TB15P | C₇H₇NO₂ carboxylate alkyl pyridine |
| TB16P | C₈H₁₆ Alkyl cyclohexane |

As shown in Table 1, the breathalyzer comprises a plurality of channels, each channel calibrated for a particular VOC.

The breath sample to be analyzed by the breathalyzer is collected as follows. A subject (for example, a breath sample donor) breathes onto the nanocarbon treated sample card for 5-10 consecutive breaths to allow alveolar breath to be captured onto an adsorber film disposed at the surface of the card. The sample card comprising the breath sample is inserted into a heated desorber inlet of the breathalyzer for rapid screening of VOCs that the breathalyzer is programmed to identify. The volatilized breath sample is introduced into a gas chromatography unit coupled to an ion mobility spectrometer (GC-IMS) for separation of VOCs and analysis and determination thereof. It is desired that, in a span of preferably no more than 20 seconds, results indicating whether the subject has an airborne infectious disease (e.g., tuberculosis) are displayed on the breathalyzer.

The GC-plasmagram profile is analyzed by a four-layer detection algorithm and an AI decision making process.

The breathalyzer employs an air sample collection approach that is relatively non-invasive in comparison to various swab collection mechanisms (e.g., nasopharyngeal) and minimizes exposure risk during sample collection, transfer, and analysis. The collection media trap exhaled breath from a person allows enrichment of five or more exhalations onto the collection media (the nanocarbon treated sample card, in this embodiment). For example, the person may deliver 5 to 15 exhalations, 5 to 12 exhalations, 5 to 10 exhalations, 5 to 9 exhalations, 5 to 8 exhalations, 5 to 7 exhalations, or 5 to 6 exhalations.

As contemplated in this embodiment, the GC component of the breathalyzer is used to separate predetermined analytes, specifically volatile organic compounds (VOCs) associated with TB, from other compounds or to separate predetermined analytes from one another. The GC acts as a pre-analysis separator so that VOCs of interest may be directed to the chemical ionization source of the IMS. When certain predetermined elution conditions have been met, there is detection of the VOCs in both positive and negative modes for molecular ions and dimers associated with the clustering of the analytes in the ionization source.

The method preferably provides the advantages associated with IMS detection, such as high sensitivity, good specificity, and fast detection rates. A major difficulty with the use of breathalyzers under field conditions is the complex chemical matrix that can be found on the breath of different people. Inter alia, sex, health condition, ingested food and drink, use of medication, and whether a person is a smoker all affect what is found on a person's breath, and there are other factors that have an effect as well. This wide variety of chemical substances can cause detector overload, potential false positives or negatives, and contamination of the system with the biological sample. VOCs on human breath are usually present in the low parts per billion concentration. A breath sample, therefore, often requires some enrichment to bring the levels to the detection limit of the detector, as mentioned above.

As contemplated in another embodiment, a filter on the sample card is used to capture the volatile components on the infected person and exclude water and other light weight gases not of interest to tuberculosis analysis, and preferably trap the target volatiles that are found on the person.

The filter may comprise a coating configured to absorb/adsorb the one or more target substances. Coating materials were disclosed in U.S. Pat. No. 9,329,156 to Nacson (incorporated herein by reference) for trapping explosive vapors and drugs. The person to be screened breathes into the filter a minimum of five or greater exhalations. The filter is then inserted into the heated desorber unit of the breathalyzer for vaporization of the sample into the separator and enrichment in the internal GC column. In turn, the GC column is thermally ramped under an internal carrier gas to provide the introduced sample into components, which are introduced into the ionization source. Positive and negative ions are formed for each species of interest, as well as dimer peaks, because of the internal ion-molecular ionization processes. The IMS detector is configured to detect the ions of the aforementioned VOCs.

The internal desorber unit is heated to 200° C. which is sufficient to destroy any biological sample that is collected on the filter. Another aspect of the invention is trapping the effluent after internal sterilization on an internal charcoal filter(s). This aspect of the invention makes the filter reusable for the next person.

As contemplated in another embodiment, there is provided a novel Retention-Time Separation-Analysis (RTCA) test-system casting substance-quantifiers as distinguishable nest-peaks. A Drift-Time-Peak-Separation (DTPS) technique is used for time-clustered structures. As contemplated in another embodiment, there is provided a combined Derivative-Based-Retention-Time-Separation-Approach (DBRTA), which allows identification of low signal-noise peaks over background baseline. Complex-cluster benchmarks with 3-4 nested peaks residing in the analyte of interest identification area with relatively high peak intensity are addressed by this novel-architecture of multi-Shard detection designed to reduce misdetection working in dual-single polarity schemes. This advancement increases resolution of nested structures characteristics of sensed complex chemical compositions.

It is contemplated that any part of any aspect or embodiment discussed in this specification can be implemented or combined with any part of any other aspect or embodiment discussed in this specification. While particular embodiments have been described in the foregoing, it is to be understood that other embodiments are possible and are intended to be included herein. It will be clear to any person skilled in the art that modification of and adjustment to the foregoing embodiments, not shown, is possible.

What is claimed is:

1. An apparatus for detecting tuberculosis infection in a subject, the apparatus comprising a breath sample receiver and a detector associated with the breath sample receiver for detecting at least one tuberculosis-infection-indicative volatile organic compound in a breath sample, the at least one tuberculosis-infection-indicative volatile organic compound selected from a group consisting of C₅H₁₀O aldehyde, C₇H₁₄O aliphatic ketone, C₉H₁₈O aliphatic ketone, C₈H₁₆O aldehyde, C₈H₁₈O alkyl alcohol, C₉H₁₂ alkyl aromatic, C₁₂H₂₆₀ alkyl alcohol, C₉H₁₈O aliphatic aldehyde, C₁₀H₁₄ alkyl aromatic, C₇H₇NO₂ carboxylate alkyl pyridine, C₈H₁₆ alkyl cyclohexane, and any combination thereof.

2. The apparatus as claimed in claim 1, wherein the breath sample receiver is capable of receiving a nanocarbon treated sample card containing the breath sample.

3. The apparatus as claimed in claim 2, wherein the nanocarbon treated sample card comprises a filter for separating the at least one tuberculosis-infection-indicative volatile organic compound in the breath sample from other volatile organic compounds that are not of interest in a determination of tuberculosis infection.

4. The apparatus as claimed in claim 3 wherein the filter is a charcoal filter.

5. The apparatus as claimed in claim 1, further comprising a gas chromatographic unit.

6. The apparatus as claimed in claim 5, wherein the detector is an ion mobility spectrometer coupled to the gas chromatographic unit.

7. The apparatus as claimed in claim 6, further comprising a retention-time separation-analysis program for analyzing the at least one tuberculosis-infection-indicative volatile organic compound in the breath sample.

8. The apparatus as claimed in claim 6, further comprising a derivative-based-retention-time separation program for analyzing the at least one tuberculosis-infection-indicative volatile organic compound in the breath sample.

9. The apparatus as claimed in claim 6, further comprising a desorber unit.

10. A method for detecting tuberculosis infection, the method comprising collecting a breath sample from a subject, and detecting in the breath sample at least one tuberculosis-infection-indicative volatile organic compounds selected from a group consisting of C₅H₁₀O aldehyde, C₇H₁₄O aliphatic ketone, C₉H₁₈O aliphatic ketone, C₈H₁₆O aldehyde, C₈H₁₈O alkyl alcohol, C₉H₁₂ alkyl aromatic, C₁₂H₂₆₀ alkyl alcohol, C₉H₁₈O aliphatic aldehyde, C₁₀H₁₄ alkyl aromatic, C₇H₇NO₂ carboxylate alkyl pyridine, C₈H₁₆ alkyl cyclohexane, and any combination thereof.

11. The method as claimed in claim 10, wherein the at least one tuberculosis-infection-indicative volatile organic compounds of the breath sample is collected on a nanocarbon treated sample card.

12. The method as claimed in claim 11, wherein the nanocarbon treated sample card comprises a filter for separating the at least one tuberculosis-infection-indicative volatile organic compound in the breath sample from other volatile organic compounds that are not of interest in a determination of tuberculosis infection.

13. The method as claimed in claim 11, further comprising inserting the nanocarbon treated sample card into the breath sample receiver of the apparatus as claimed in claim 1, and separating the at least one tuberculosis-infection-indicative volatile organic compounds by gas chromatography.

14. The method as claimed in claim 13, further comprising detecting the at least one TB-infection-indicative volatile organic compounds by an ion mobility spectrometer.

15. The method as claimed in claim 14, further comprising analyzing the at least one TB-infection-indicative volatile organic compounds detected by the ion mobility spectrometer by using one of a retention-time separation-analysis program and a derivative-based-retention-time separation program.

16. The method as claimed in claim 14, further comprising comparing the profile of the at least one TB-infection-indicative volatile organic compounds detected by the ion mobility spectrometer against a chemical profile from a library that is pre-installed on the apparatus
